(19) United States
(12) Patent Application Publication
(10) Pub. No.: US 2005/0100516 A1
(43) Pub. Date: May 12, 2005

(54) Hydroalcohol based self-tanning composition

(75) Inventor: Thomas W. Dann, Oldsmar, FL (US)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Formulated Solutions, LLC

(21) Appl. No.: 10/703,241

(22) Filed: Nov. 7, 2003

(51) Int. Cl. A61K 7/42; A61K 7/021

(52) U.S. Cl. 424/59

(57) ABSTRACT

The invention is a sunless tanning composition that can be applied to the skin as an aerosol. The composition is a hydroalcohol system that contains at least one self-tanning agent and a surfactant. The surfactant reduces the composition's surface tension so that it is applied to the skin in a uniform and even manner. As a result, the composition helps eliminate problems associated with most sunless tanning products.

### BACKGROUND OF THE INVENTION

Artificial tanning products, also known as sunless tanning products, offer an alternative to natural or artificial sunlight for achieving a natural tanned appearance. Sunless tanning products typically contain an active ingredient, such as a reducing sugar or colorant, which causes the skin to assume a browned appearance. The most popular active ingredient is DHA. DHA is a reducing sugar that interacts with the dead surface cells found in the epidermis, or the outermost layer of the skin, to produce a color change.

Most sunless tanning products come in the form of an oil, lotion, or gel. These lotions are typically an emulsion between an oil phase and a water phase. They normally require the user to apply the lotion to the body using the hands to spread the lotion over the body.

Problems often associated with sunless tan products include uneven application and streaking. If too much, or too little, lotion is applied in a single area, the area may become darker or lighter than the surrounding skin. As a result, the skin coloring may appear unnatural, streaked, or splotchy. Most sunless tanning products are applied using the hands. This will often cause the user's palms to be exposed to the active ingredients for a longer period of time than the rest of the body. This may result in the palms having an appearance that is significantly darker than desired. Other problems associated with using self-tanning products include a general feeling of greasiness and the mess of applying the product. Although self-tanning aerosols have been proposed, aerosols typically do not solve the problems associated with self-tanning products.

Thus, there still exists a need for a sunless tanning product that is easily applied, covers the skin in a uniform and even manner, and does not require manual application using the hands.

### BRIEF SUMMARY OF THE INVENTION

The invention is a sunless-tanning composition that substantially reduces problems of uneven application, streaking, and the like. The self-tanning composition can be applied as an aerosol.

The composition is a hydroalcoholic system that contains at least one self-tanning agent and a surfactant. The hydroalcoholic system is a carrier for the active ingredients and the surfactant. The hydroalcoholic carrier is a combination of water and an alcohol, such as ethanol. Typically the active ingredient is a reducing sugar, such as DHA.

The surfactant reduces the composition's surface tension. A reduction in surface tension greatly improves product application in two ways. It aids in the atomization of the hydroalcholic medium so that the composition can be released as a fine spray. After the composition is released and applied to the skin, the reduction in surface tension helps prevent the composition from beading or pooling on the skin's surface. As a result, the composition is deposited onto the skin as a uniform thin layer. This aids in the formation of a browned and tanned appearance that is more even and has minimal, if any, streaking or blotchiness.

The solvent acts as a carrier for the tanning agents and promotes rapid drying of the composition on the skin. The composition can be delivered as an aerosol that is comprised of a small particle spray. A propellant is used to atomize and deliver the composition to the skin as an aerosol.

### DETAILED DESCRIPTION OF THE INVENTION

The invention now will be described more fully. The invention may be embodied in many different forms and should not be construed as limited to the embodiments set forth herein; rather, these embodiments are provided so that this disclosure will satisfy applicable legal requirements.

The invention is a self-tanning composition that can be applied to the skin in the form of an aerosol spray. The composition contains at least one-self tanning agent and a surfactant. Delivering the composition in the form of an aerosol allows for wider and fuller coverage, and helps to eliminate many of the problems associated with self-tanning compositions. The surfactant reduces the composition's surface tension. As a result, the composition is spread more evenly over the skin's surface. Using an aerosol delivery system also helps to eliminate streaking that is often associated with the uneven application of sunless tanning products.

The self-tanning agents are generally selected from among mono- or polycarbonyl compounds. Useful compounds include isatin, alloxan, ninhydrin, glyceraldehydes, mesotartaric, aldehyde, glutaraldehyde, erythrulose, and dihydroxyacetone (DHA).

DHA is a particularly useful self-tanning agent. DHA is a 3-carbon sugar that when applied to the skin causes a chemical reaction with amino acids in the surface cells of the skin producing a darkening effect. Other reducing sugars that have self-tanning properties may be substituted, although not necessarily with equivalent results. These include, for example, glucose, xylose, fructose, reose, ribose, arabinose, allose, tallose, altrose, mannose, galactose, sucrose, and lactose.

The percent concentration of active ingredients used will vary depending upon the strength of the particular formulation. For instance, the composition could be available in Light, Medium and Dark formulations. Typically, DHA will be present in a percent concentration that is from about 1 to 25% by weight of the composition. To achieve the best results, DHA will be present in an amount that is from about 2 to about 8%, and some what better is a concentration that is about 3 to 5% by weight. Typically, a DHA concentration greater than 25% offers no additional benefits.

U.S. Pat. No. 6,451,293, which is hereby incorporated by reference, discloses that combining erythrulose with a reducing sugar, such as DHA, provides an even and longer lasting tanned appearance. While not wishing to be bound by theory, it is believed that erythrulose penetrates deeper into the skin than DHA and is able to interact with amino acids in lower layers.

Erythrulose, also known as (S,R)-2,3,4-trihydrocybutanal, can be used in either the D or L-form or as a racemate. In compositions containing both erythrulose and DHA, erythrulose is blended in a mixture with DHA in a ratio that is from about 1 to 50%. Typically, the amount of erythrulose present is from about 0 to 12% by weight. In concentrations exceeding 12% odor becomes more noticeable and the composition's viscosity is adversely affected. Preferably, the amount of erythrulose is from about 0.75 to 6%, and somewhat more preferably from about 1 to 1.75%.

It is difficult to atomize water in an aerosol. When water is atomized in an aerosol spray, the resulting particles have a tendency to be wet and coarse. This could cause the composition to bead or clump on the skin resulting in an uneven or blotchy appearance.

It has been found that the addition of a surfactant greatly improves the performance of the composition, especially when it is delivered as an aerosol. The surfactant helps solubilize the water in a propellant and reduces the composition's surface tension. A reduction in surface tension allows a propellant to effectively atomize the composition so that the composition forms an aerosol having fine particles. After applying the composition on the skin, the surfactant improves the flow and distribution of the composition on the skin. In absence of the surfactant, the composition would have a tendency to bead or flow together. The surfactant ensures that the composition is applied to the skin in a uniform, almost sheet like, manner.

Typically, after atomization, individual particle size should be from about 10 to 50 microns, and somewhat more typically, particle size is from about 10 to 35 microns. Particle sizes below 10 microns are inhalable and should therefore be avoided. Particle sizes greater than 50 microns are usually too course and do not spread evenly.

The surfactant should be miscible in water and the hydroalcohol solvent. Preferably, the surfactant is a polyhydric alcohol, such as dipropylene glycol or ethoxydiglycol. Typically, dipropylene glycol is present in an amount that is less than 5% by weight of the composition. Best results are achieved when the concentration of dipropylene glycol is from about 2 to 3% by weight. Concentrations up to 25% by weight can be used, although not necessarily with equivalent results.

Optionally, additional surfactants can be used in combination with dipropylene glycol to help stabilize the propellant in the hydroalcohol system and to improve composition flowability on the skin. Additional useful surfactants, include without limitation, polysorbate 20, ethoxydiglycol, and carbitol, and blends thereof. When present, these surfactants are usually in an amount from about 1 to 5% by weight.

The composition is a single phase homogeneous system. The composition contains a hydroalcohol solvent that is miscible in water and serves as the carrier for the active ingredients. The hydroalcohol system is substantially emulsion free. Substantially emulsion free means that although the system is primarily comprised of ingredients that are soluble in water and alcohol, it may also contain small quantities of oils, fragrances, or emulsifiers. If non-hydroalcohol ingredients are included, they are typically present in amount that is less than 2% by weight of the total composition, and even more typically less than 1%.

The hydroalcohol system is typically comprised of a carrier for the actives and purified water. The carrier is typically a fast drying solvent. The solvent serves several useful functions. It is a carrier for the actives, cools the skin, and promotes rapid drying of the skin. Rapid drying helps to ensure that the thin composition layer is applied evenly to the skin without streaking or blotchiness.

Alcohol is an excellent solvent for facilitating the delivery of a wide variety of cosmetic and pharmaceutical actives, and because it readily evaporates after facilitating the delivery of active components. In particular, alcohol is an especially useful solvent in the invention because it helps stabilize any propellants that may be present. Typical alcohols include isopropyl alcohol, ethanol, or anhydrous ethyl alcohol, or blends thereof. The alcohol may be pure alcohol or a denatured alcohol such as, SD Alcohol 23-A, SD Alcohol 40, and SD Alcohol 40-B. Other grades of alcohol may be used provided that they are cosmetically or pharmaceutically acceptable and can be used in the compositions of the invention. The solvent is typically present in an amount that is from 1 to 75% by weight relative to the total weight of the composition. Somewhat more typical, is a concentration percentage that is from about 45 to 50% by weight.

The concentration of pure water will preferably be from about 11 to 20% by weight. The water concentration can range from about 2 to 50%, although the composition may be adversely affected by concentrations greater and lower than these values.

DHA works best in an acidic environment having a pH that is from about 4.5 to 5.5. Typically, an acidifying agent, such as citric acid, acetic acid, dilute hydrochloric acid, or the like, is used to adjust the composition's pH. The acidifying agent is added until the pH is within the desired range.

Aerosol propellants that are useful in the invention typically have a boiling point within the range of from about −45 degree C. to about 5 degree C. The aerosol propellants are liquefied when packaged in conventional aerosol containers under pressure. The rapid boiling of the aerosol propellant upon exiting the aerosol container aids in the atomization of the active ingredients.

Aerosol propellants useful in the present invention include those well known in the cosmetology and dermatology industry, such as, for example, the chemically-inert hydrocarbons such as propane, n-butane, isobutane and cyclopropane, and mixtures thereof, as well as halogenated hydrocarbons such as dichlorodifluoromethane (propellant 12) 1,1-dichloro-1,1,2,2-tetrafluoroethane (propellant 114), 1-chloro-1,1-difluoro-2,2-trifluoroethane (propellant 115), 1-chloro-1,1-difluoroethylene (propellant 142B), 1,1-difluoroethane (propellant 152A), monochlorodifluoromethane, and dimethyl ether, and mixtures thereof. Isobutane, used singly or mixed with other hydrocarbons, particularly propane (propellant A-46), is preferred for use in the aerosol. Other hydrocarbon propellants may be used, although not necessarily with equivalent results.

The amount of hydrocarbon propellant that can be present in the composition is typically from about 5 to 80% by weight, relative to the entire weight of the composition, with a range from about 20 to 30% providing better results, and about 25% giving the best results. At concentrations below 5%, atomization of the composition may be adversely affected.

Compressed gas, used by itself or in combination with hydrocarbons or halogenated hydrocarbons, may also be used as a propellant. Typical compressed gasses include, carbon dioxide, nitrogen, and nitrous oxide, and mixtures thereof. If compressed gas is used, the concentration is typically between 1 and 4% by weight. At concentrations exceeding 10%, the compressed gas is usually no longer soluble in the hydroalcohol medium. In a blended state, the compressed gas/hydrocarbon blend can have a concentration ranging from about 0.5 to 90%, with a concentration from about 20 to 25% providing the best results.

The composition may also be delivered by a mechanical propellant or self-pressurization rather than using a chemical propellant. For instance, the composition could be contained in a canister that can be manually pumped to create air pressure to expel and atomize the ingredients. Alternatively, the composition can be contained in a pouch or bladder that expands during filling. After filling, external pressure can be applied to the pouch or bladder to create an outward force. The outward force expels the composition from the pouch or bladder.

The composition may also contain additional optional ingredients such as fatty substances, organic solvents, thickeners, softeners, moisturizers, antioxidants, agents for combating free radicals, opacifiers, stabilizers, emollients, silicones, alpha-hydroxy acids, antifoaming agents, vitamins, insect repellants, anti-inflammatories, fragrances, preservatives, fillers, polymers, basifying or acidifying agents, colorants, or any other ingredient that is cosmetically or pharmaceutically acceptable, depending upon the intended use.

Typically, the composition will contain a fragrance to mask any smell that may be associated with the active ingredients. Ordinone™ available from Belle Aire Fragrances, is a particularly useful mal odor absorbing compound that helps to neutralize any unpleasant odors associated with DHA. Zinc rincinoleate compounds are also useful to counteract unpleasant odors, although not necessarily with equivalent results. The composition may also contain minute quantities of other fragrances to give the composition a pleasant smell. If other fragrances are used, it is important that they do not adversely interfere with Ordinone's odor neutralizing abilities.

The composition may also contain dimethyl isosorbide. Dimethyl isosorbide is a water soluble solvent that has skin penetrating properties. It helps the self-tanning agent penetrate into the skin and improves the composition's spreading characteristics on the skin. When present, dimethyl isosorbide is typically in a concentration range from about 0.5 to 10% by weight.

The composition may also contain water soluble humectants. Because DHA is very drying to the skin, a humectant may be added to keep the skin hydrated and to improve skin feel. Typical humectants include polyhydric alcohols such as, glycerin, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. Other useful humectants include high fructose corn syrup, corn syrup, and the like. For best results, the humectant is preferably glycerin or sorbitol. When present, the amount of humectant may range anywhere from 0.5 to 25%, preferably between 1 and 15% by weight of the composition.

The invention also relates to treatment method for artificially tanning or browning the skin. The treatment method consists of applying to the skin an effective amount of the composition defined above. The method also includes the step of positioning an aerosol container containing the composition a distance from the skin that delivers an effective amount of the composition in an even and uniform manner. To achieve the best results, the aerosol container is typically positioned about 6 to 12 inches from the skin.

### EXAMPLE

| AEROSOL COMPOSITION | % BY WEIGHT |
|---|---|
| Purified Water | 11.88 |
| Dihydroxyacetone | 3.75 |
| Erythrulose | 1.5 |
| Dipropylene Glycol | 2.25 |
| Ethoxydiglycol | 3.22 |
| Propylene Glycol | 1.5 |
| Dimethyl Isosorbide | .75 |
| Fragrance | .6 |
| Polysorbate 20 | .23 |
| Glycerin | .38 |
| Sorbitol | 0.08 |
| Fragrance | .11 |
| Color Solution | q.s. |
| SDA-40B | 48.75 |
| Citric acid | q.s. to pH 4.5-5.5 |
| Propellant A-46 | 25 |

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A composition for giving the skin an artificial coloration comprising:
a) at least one self-tanning agent;
b) a surfactant; and
c) a hydroalcohol.

2. The composition according to claim 1, wherein the at least one self-tanning agent is a mono- or polycarbonyl compound.

3. The composition according to claim 1, wherein the self-tanning agent is isatin, alloxan, ninhydrin, glyceraldehydes, mesotartaric, aldehyde, glutaraldehyde, erythrulose, a pyrazoline-4,5-dione derivative, dihydroxyacetone (DHA), or a 4,4-dihydroxypyrazoline-5-dione derivative.

4. The composition according to claim 1, wherein the concentration of the self-tanning agent is from about 1 to 25% by weight relative to the total weight of the composition.

5. The composition according to claim 1, wherein the self-tanning agent is DHA.

6. The composition according to claim 1, wherein the concentration of the surfactant is less than 25% by weight relative to the weight of the composition.

7. The composition according to claim 1, wherein the surfactant is selected from the group consisting of diproplyene glycol, polysorbate 20, ethoxydiglycol, and carbitol.

8. The composition according to claim 1, wherein the hydroalcohol is comprised of purified water and an alcohol solvent.

9. The composition according to claim 1, wherein the alcohol solvent is selected from the group consisting of anhydrous ethyl alcohol, isopropyl alcohol, ethanol, SD Alcohol 23-A, SD Alcohol 40, and SD Alcohol 40-B, and blends thereof.

10. The composition according to claim 8, wherein the concentration of the alcohol solvent is from about 1 to 75% by weight relative to the weight of the composition.

11. A composition for giving the skin an artificial coloration comprising:
a) at least one self-tanning agent;
b) a surfactant that is less than 5% by weight relative to the weight of the composition; and
c) a hydroalcohol.

12. The composition according to claim 11, wherein the self-tanning agent is a combination of DHA and erythrulose.

13. The composition according to claim 11, wherein the concentration of DHA is from about 3 to 5% and the concentration of erythrulose is from about 1 to 2% by weight relative to the total weight of the composition.

14. The composition according to claim 12, wherein the concentration of DHA is about 3.75% and the concentration of erythrulose is about 1.5% by weight relative to the total weight of the composition.

15. The composition according to claim 11, wherein the surfactant is dipropylene glycol.

16. The composition according to claim 15, wherein the concentration of dipropylene glycol is from about 2 to 3% by weight relative to the total weight of the composition.

17. The composition according to claim 11, wherein the hydroalcohol is a combination of a solvent and purified water.

18. The composition according to claim 13, wherein the solvent is selected from the group consisting of anhydrous ethyl alcohol, isopropyl alcohol, ethanol, SD Alcohol 23-A, SD Alcohol 40, and SD Alcohol 40-B, and blends thereof.

19. The composition according to claim 17, solvent is from about 1 to 75% by weight relative to the total weight of the composition.

20. The composition according to claim 17, wherein the concentration of the solvent is from about 45 to 50% by weight relative to the total weight of the composition.

21. An aerosol composition for giving the skin an artificial coloration comprising:
a) at least one self-tanning agent;
b) a surfactant that is less than 5% by weight, relative to the weight of the composition;
c) a propellant; and
d) a hydroalcohol

22. The composition according to claim 21, wherein the propellant is a hydrocarbon selected from the group consisting of isobutane, propane, n-butane, and mixtures thereof.

23. The composition according to claim 21, wherein the concentration of the propellant is from about 5 to 80% by weight relative to the weight of the total composition.

24. The composition according to claim 22, wherein the concentration of the propellant is about 25% by weight relative to the weight of the total composition.

25. The composition according to claim 21, wherein the propellant is a hydrocarbon, compressed gas, or a halogenated hydrocarbon, or a mixture thereof.

26. The composition according to claim 25, wherein the propellant is selected from the group consisting of dichlorodifluoromethane (propellant 12) 1,1-dichloro-1,1,2,2-tetrafluoroethane (propellant 114), 1-chloro-1,1-difluoro-2,2-trifluoroethane (propellant 115), 1-chloro-1,1-difluoroethylene (propellant 142B), 1,1-difluoroethane (propellant 152A), monochlorodifluoromethane, and dimethyl ether, and mixtures thereof.

27. The composition according to claim 25, wherein the propellant is a compressed gas selected from the group consisting of carbon dioxide, nitrogen, and nitrous oxide, and mixtures thereof.

28. A composition according to claim 25, wherein the propellant is a blend of a hydrocarbon and a compressed gas.

29. A composition according to claim 28, wherein the concentration of the propellant is from about 20 to 25% by weight relative t the weight of the concentration.

30. A composition according to claim 25, wherein the propellant is a blend of a halogenated hydrocarbon and a compressed gas.

31. The composition according to claim 21, wherein the hydroalcohol is a combination of purified water and a solvent.

32. The composition according to claim 31, wherein the solvent is an alcohol selected from a group consisting of SD Alcohol 23-A, SD Alcohol 40, and SD Alcohol 40-B.

33. The composition according to claim 21, wherein the composition includes citric acid.

34. The composition according to claim 21, wherein the composition has a pH from about 4.5 to 5.5.

35. The composition according to claim 31, wherein the concentration of the water is from about 10 to 15% by weight relative to the total weight of the composition.

36. An aerosol composition for giving the skin an artificial coloration comprising:
a) a self-tanning agent that is a combination of DHA and erythrulose, wherein the concentration of DHA is from about 3 to 5% and the concentration of erythrulose is from about 1 to 1.75% by weight relative to weight of the composition;
b) dipropylene glycol that is from about 2 to 3% by weight relative to the weight of the composition;
c) a hydrocarbon propellant that is from about 20 to 30% by weight relative to the weight of the composition; and
d) a hydoalcohol comprised of a solvent and purified water that is about 45 to 50% by weight relative to the weight of the composition, and wherein the aerosol particles have a size from about 10 to 50 microns.

37. The composition according to claim 36, wherein the ratio of DHA to erythrulose is from 1:0 to 1:0.5

38. The composition according to claim 36, wherein the concentration of dipropylene glycol is about 2.25% by weight relative to the total weight of the composition.

39. The composition according to claim 36, wherein the propellant is a blend of propane and isobutane.

40. The composition according to claim 36, further comprising a mal odor neutralizer.

41. The composition according to claim 40, wherein the mal odor neutralizer is Ordinone.

42. The composition according to claim 36, further comprising a humectant selected from the group consisting of sorbitol and glycerin.

43. The composition according to claim 36, further comprising at least one cosmetic adjuvant that is a fragrance, emulsifier, solvent, moisturizer, fatty substance, a softener, an antioxidant, a stabilizer, an emollient, an insect repellant, colorants, UV protectant agent, a preserving agent, a filler, or anti-inflammatory agent.

44. The composition according to claim 36, further comprising dimethyl isosorbide.

45. The composition according to claim 44, wherein the concentration of dimethyl isosorbide is from about 0.05 to 10% by weight relative to the total weight of the composition.

46. The composition according to claim 36, wherein the composition is substantially emulsion free.

47. The composition according to claim 36, wherein the aerosol particles have a size from about 10 to 35 microns.

48. A cosmetic process for tanning or browning the skin, comprising applying to the skin an effective amount of the cosmetic composition according to claim 1.

49. A method of artificially tanning or browning the skin, comprising the steps of:
a) combining at least one skin-tanning agent with a surfactant in a hydroalcohol carrier;
b) delivering the skin-tanning agent and surfactant combination to the skin in the form of an aerosol; and
c) allowing the composition to dry for an effective amount time to tan or brown the skin.
